(19) United States
(12) Patent Application Publication
(10) Pub. No.: US 2023/0172978 A1
(43) Pub. Date: Jun. 8, 2023

(54) USE OF EXTRACELLULAR VESICLES AS IMMUNOPROPHILACTICS AND IMMUNOTHERAPEUTICS FOR LEISHMANIASIS

(71) Applicant: YEDITEPE UNIVERSITESI, Istanbul (TR)

(72) Inventors: Fikrettin SAHIN, Istanbul (TR); Zeynep ISLEK, Istanbul (TR); Batuhan Turhan BOZKURT, Istanbul (TR); Oguz Kaan KIRBAS, Istanbul (TR); Mehmet Hikmet UCISIK, Istanbul (TR)

(73) Assignee: YEDITEPE UNIVERSITESI, Istanbul (TR)

(21) Appl. No.: 17/923,244

(22) PCT Filed: May 4, 2021
(86) PCT No.: PCT/TR2021/050425

§ 371 Date: Nov. 4, 2022

(30) Foreign Application Priority Data
May 4, 2020 (TR) 2020/06914

(51) Int. Cl. A61K 35/15; A61P 33/00

(52) U.S. Cl. CPC A61K 35/15; A61P 33/00; A61K 35/74

(57) ABSTRACT

A use of extracellular vesicles obtained from macrophages infected with parasites as immunotherapeutic and prophylactic agents in the disease Leishmaniasis is disclosed. The objective of the invention is the use of a drug formulation, which exhibits almost complete activity on Leishmania parasites and infected cells within 72 hours, but does not have any show side effects on healthy cells, in the treatment and prophylaxis of Leishmaniasis.

### CROSS REFERENCE TO THE RELATED APPLICATIONS

This application is the national phase entry of International Application No. PCT/TR2021/050425, filed on May 4, 2021, which is based upon and claims priority to Turkish Patent Application No. 2020/06914, filed on May 4, 2020, the entire contents of which are incorporated herein by reference.

### TECHNICAL FIELD

The present invention relates to use of extracellular vesicles obtained from macrophages infected with parasites as immunotherapeutic and prophylactic agents in the disease Leishmaniasis.

### BACKGROUND

Leishmaniasis is a common name given to a group of vector-borne diseases that are transmitted to humans by the bite of female sandflies infected with Leishmania protozoan parasites. Accordingly, the parasite, which is in amastigote form when it infects the sandfly, develops as promastigote in the digestive system of the fly and is transmitted to the animal or human that the fly bites as promastigote. Promastigote infects macrophages in the body and causes disease upon turning into amastigotes in the macrophages.

According to the World Health Organization, leishmaniasis is widely seen in more than 60 countries worldwide especially in the Southern European, Middle Eastern and North African countries, including Turkey and the surrounding geography. Visceral Leishmaniasis (VL) known as a type of leishmaniasis, colloquially known with the local name Kala Azar Disease, can be fatal within two years if it is not treated. Leishmania infantum is the main parasite species that causes this disease in the geography of our country and can cause VL.

As with other parasitic diseases, chemotherapy is the most effective method in the treatment of leishmaniasis. However, the high toxicity values of the antiparasitic compounds and the resistance developed by the parasites against the drug over time limits the applicability of chemotherapy. Improving conventional therapy in such infections significantly requires more effective and selective drugs or drug formulations with low toxicity. The inadequacy of treatment methods has forced scientists to try new methods in the field of leishmaniasis. Among these trials, exosomes yield effective results.

Extracellular vesicles are small sacs which are involved in intercellular transport of substances and are separated by at least one lipid bilayer from the cytoplasm fluid. Exosomes, which are one of the extracellular vesicles, are vesicles which are released by many organisms from prokaryotes to high eukaryotes and plants, and which contain lipid bilayer membranes of different sizes. The importance of these vesicles lies behind the capacity of transferring information to the other cells in order to influence the cell function. Signal transfer via exosomes is carried out by means of biomolecules in many different categories consisting of proteins, lipids, nucleic acid and sugars. Because they carry the surface proteins of the cell from which they are produced, exosomes target the type of cell in which they are delivered in in vivo systems. These properties make exosome suitable for carrying nucleic acids for drug, bioactive substance and gene therapy. Another distinctive aspect of the exosomes is that they are particular or specific to the cell wherein the signals and cargos they carry are produced and to the current physiological conditions of the cell. Exosomes of different organisms, exosomes of different types of cells of the same organism, and exosomes of the same cell in different conditions show different properties.

Each cell produces exosomes for its own purposes. The use of exosomes, which are involved in signaling pathways, in immunotherapy has recently emerged as a new method in researches on diseases targeting particularly the immune system. Within the scope of these studies, exosomes isolated from different cell groups (eukaryotes and prokaryotes) are used to regulate the immunological response. In parasitic infections the role played by the exosomes, which are produced by pathogenic microorganisms, on the onset and progression of the infection and their interaction with the host immune cells have been recently used for the treatment and prevention of infectious diseases.

The Chinese patent document no. CN109890964, an application known in the art, discloses extracellular vesicles and their use for the delivery of therapeutic agents to ocular tissues for the treatment of ophthalmic diseases.

The Chinese patent document no. CN1646147A, an application known in the art, discloses methods and compositions for targeting of a systemically generated immune response to a specific organ or tissue.

The International patent document no. WO2018101782, an application known in the art, discloses an exosome for stimulating T cells and the pharmaceutical use thereof.

The International patent document no. WO201389738, an application known in the art, discloses a method of producing exosomes which are produced by being isolated from an antigen presenting cell, such as a dendritic cell, B lymphocyte, or macrophage and which can be used in the treatment of Leishmaniasis.

As with many other parasitic diseases, chemotherapy is the most effective method in the treatment of leishmaniasis. However, the high toxicity values of the antiparasitic compounds and the resistance developed by the parasites against the drug over time limits the applicability of chemotherapy. Improving conventional therapy in such infections significantly requires more effective and selective drugs or drug formulations with low toxicity.

Particularly the treatment of the disease Kala azar (VL) is largely based on pentavalent antimonials. The main problem limiting the success of this chemotherapy is the increasing resistance of parasites against antimonials. In addition, antimonials are toxic, have strong side effects and require long-term hospitalization of the patient.

In the treatment of leishmaniasis, pentavalent antimonials (pentostam and glucanthime), miltefosine, paromomycin, amphotericin formulated with deoxycholic acid (Fungizone) and amphotericin formulated with liposomes (AmBisome) are used. However, high costs and toxic side effects appear as difficulties limiting the treatment.

### SUMMARY

The objective of the present invention is the use of a drug formulation, which exhibits almost complete activity on Leishmania parasites and infected cells within 72 hours, but does not have any show side effects on healthy cells, in the treatment and prophylaxis of Leishmaniasis.

Another objective of the invention is to observe the effect of the therapy based on cellular targeting by detecting plant-derived exosomes as biocompatible vesicular systems as part of the mononuclear phagocytic system.

A further objective of the invention is loading active substances on exosomes by using the drug loading capacity of exosomes, and thus, by carrying specific drug to the target cell and thereby enhancing bioavailability of the drug, achieving the desired effect in the tumor specific target region.

Another objective of the invention is to prevent direct contact of the drug with the macrophage surface upon targeted delivery of anti-protozoan drugs to parasite infected macrophages via exosomes, and to observe an improvement in anti-parasitic activity and a decrease in toxicity in macrophages with the drug acquiring selectivity against parasites.

### BRIEF DESCRIPTION OF THE DRAWINGS

FIG. 1 is a view of the light microscope image showing the parasite infection after 5 hours following incubation of the macrophage cells in a culture medium with the parasites.

FIG. 2 is a graphical representation of the effect of the Amphotericin B-loaded infected macrophage exosomes on the viability of the Leishmania infantum parasites when administered for 72 hours in 8 different doses.

FIG. 3 is a graphical representation of the effect of the Amphotericin B-loaded infected macrophage exosomes on the viability of J774 macrophage cells when administered for 24, 48 and 72 hours in 5 different doses.

FIG. 4 is a graphical representation of the effect of the Amphotericin B-loaded infected macrophage exosomes on the viability of the macrophage cells infected with Leishmania infantum parasites when administered for 72 hours in 5 different doses.

FIG. 5 is a graphical representation of the infection rates of macrophage cells infected with Leishmania infantum following 24 hours after exosomes obtained from infected macrophages were administered for 72 hours in 5 different doses.

### DETAILED DESCRIPTION OF THE EMBODIMENTS

The present invention is the use of extracellular vesicles obtained from macrophages infected with parasites as immunotherapeutic and prophylactic agents in the disease Leishmaniasis, and the figures related to the invention are defined below:

FIG. 1 is a view of the light microscope image showing the parasite infection after 5 hours following incubation of the macrophage cells in a culture medium with the parasites.

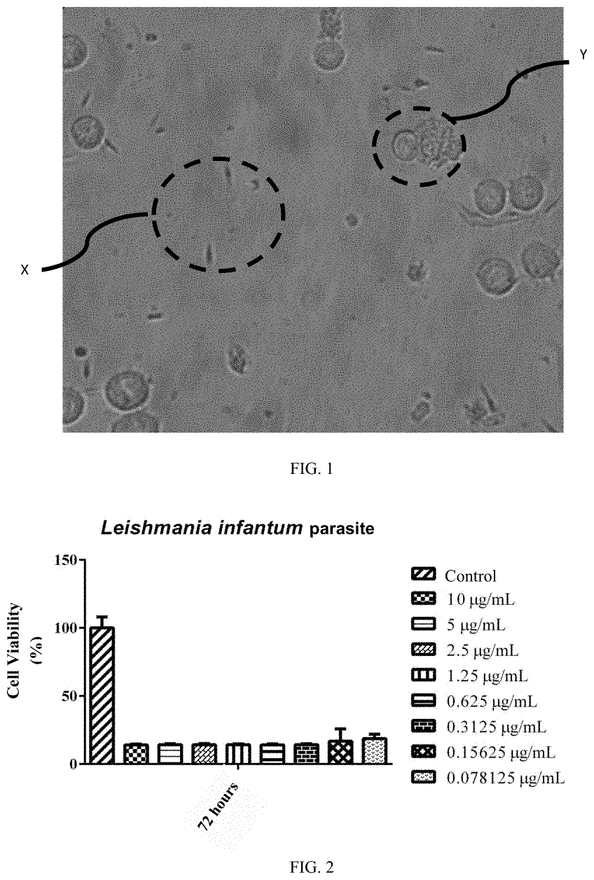

FIG. 2 is a graphical representation of the effect of the Amphotericin B-loaded infected macrophage exosomes on the viability of the Leishmania infantum parasites when administered for 72 hours in 8 different doses.

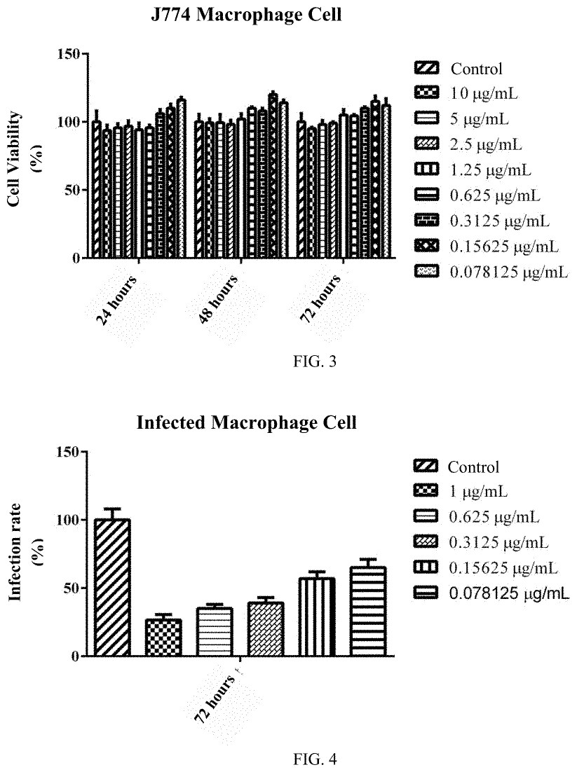

FIG. 3 is a graphical representation of the effect of the Amphotericin B-loaded infected macrophage exosomes on the viability of J774 macrophage cells when administered for 24, 48 and 72 hours in 5 different doses.

FIG. 4 is a graphical representation of the effect of the Amphotericin B-loaded infected macrophage exosomes on the viability of the macrophage cells infected with Leishmania infantum parasites when administered for 72 hours in 5 different doses.

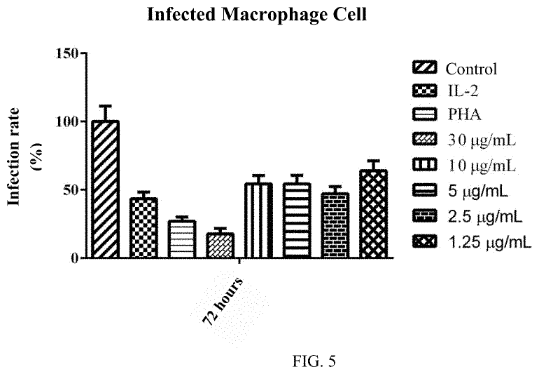

FIG. 5 is a graphical representation of the infection rates of macrophage cells infected with Leishmania infantum following 24 hours after exosomes obtained from infected macrophages were administered for 72 hours in 5 different doses.

Z. Leishmania infantum parasites

AA. Macrophage cells infected with parasites

By means of the invention, exosomes, which are a cellular extracellular vesicle system and are involved in signaling pathways, can provide a new treatment method by acting as immunotherapeutics that stimulate the immune system against Leishmania parasites that target immune system cells. Exosomes to be isolated from macrophage cells infected with Leishmania parasites are used to provide immunomodulatory activity on macrophage cells, and are an immunotherapy method alternative to existing chemotherapy methods which are inadequate in treatment.

In the prophylaxis phase; it was observed that after the macrophage cells (Y) treated with extracellular vesicles obtained from macrophages infected with parasites were infected with Leishmania parasite, the infection rate in macrophage cells (Y) decreased.

During the immunotherapy phase, the extracellular vesicles of macrophages infected with parasites were loaded with amphotericin B and the exosomes were used as a drug delivery system. It was observed that amphotericin B-loaded extracellular vesicles were highly lethal to Leishmania parasites and infected macrophage cells, however they had virtually no side effects on healthy macrophage cells (X).

Infected cell exosomes increase the body's immunity against Leishmania antigen while also preventing possible antigen-induced reactions in the body since they are biocompatible.

A method of obtaining extracellular vesicles obtained from macrophages infected with parasites for use as immunotherapeutic and prophylactic agents in the disease Leishmaniasis, comprising the steps of
preparing the parasite culture/making parasites infective,
preparing macrophage culture,
infecting macrophage culture cells with (Leishmania infantum) parasites,
isolating the extracellular vesicles obtained from infected macrophage cells via two-phase liquid system,
loading amphotericin B on exosomes obtained from macrophages infected with (Leishmania infantum) parasites and encapsulating thereof,
designating the amount of encapsulated substance,
performing resazurin assay in order to determine the effect of the promastigotes of amphotericin B-loaded exosomes on proliferation,
treating the infected macrophages with Amphotericin B-loaded infected macrophage exosomes,
treating the macrophages with the macrophage exosomes infected with the parasites and then infecting them with parasites,
determining the infection rate,
obtaining the macrophages infected with parasites as the final product.

Extracellular vesicles which are to be used in infection of macrophages and which are derived from at least one parasite selected from a group comprising the following species: Leishmania spp. (L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, L. venezuelensis), Plasmadium spp. (P. falciparum, P. vivax, P. ovale), Schistosoma spp., Toxoplasma spp. (Toxoplasma gondii), Trypanosoma brucei ssp.

The extracellular vesicles obtained from the macrophages infected with parasites are isolated by means of at least one of the following methods: isolation with aqueous two-phase systems (ATPS), graduated centrifuge, ultracentrifuge, sucrose gradient ultracentrifuge, polymeric precipitation, ultrafiltration, isolation with chromatographic methods (affinity chromatography (antibody and peptide affinity), size separation chromatography (size exclusion chromatography)), isolation with microbeads and precipitation according to ionic charge (electrical charge-based precipitation) and salting.

A pharmaceutical composition containing extracellular vesicles obtained from macrophages infected with parasites comprises at least one nano-carrier system selected from a group comprising emulsion systems, biological and chemical nanoparticles (polymeric nanoparticles, solid lipid nanoparticles), inorganic nanoparticles (metallic nanoparticles), lipid vesicular systems (liposomes, niosomes and ethosomes), dendrimers, polymer-drug conjugates, micelles and carbon nanotubes.

The pharmaceutical composition of the present invention comprises at least one active compound selected from a group comprising active compounds showing antiparasitic and/or antineoplastic activity, and binary and ternary combinations thereof as an active substance. At least one agent selected from a group comprising nitazoxanide, melarsoprol, eflornithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, ornidazole, paromomycin sulfate, pentamidine, pirimethamine, tinidazole, albendazole, mebendazole, thiabendazole, fenbendazole, triclabendazole, flubendazole, abamectin, diethylcarbamazine, suramin, pyrantel pamoate, levamisole, niclosamide, nitazoxanide, oxyclozanide, monepantel, derquantel, amphotericin B, urea stibamine, sodium stibogluconate, meglumine antimoniate, paromomycin, fluconazole, and binary or ternary combinations and encapsulations thereof is used as an active compound showing antiparasitic activity. In the pharmaceutical composition of the present invention, at least one agent selected from a group comprising cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozocin, fludarabine, raltitrexed, actinomycin D, dactinomycin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, and binary or ternary combinations and encapsulations thereof is used as an active compound showing antineoplastic activity in combination with extracellular vesicles and/or nano-carrier systems. The pharmaceutical composition of the present invention comprises extracellular vesicles obtained from macrophages infected with parasites in combination with at least one of aluminum hydroxide, aluminum phosphate, tocopherol emulsion systems containing 3D-MPL, cholesterol, CG oligonucleotide; or combinations of two or more of them.

At least one method of administration selected from a group comprising parenteral, intravenous, intradermal, subcutaneous, intraperitoneal, topical, intrathecal, intranasal, intracerebroventricular, ocular, vaginal, urethral, transdermal, sublingual, subarachnoid, rectal, periodontal, perineural, peridural, periarticular, oral, intratympanic, intratumor, intrapulmonary, intrasynovial, intramuscular, intraovarian, intrameningeal, intracorporus cavernosum, intracoronary, intracerebral, epidural, cutaneous, buccal, dental is used as the method of administration of the pharmaceutical composition of the present invention for treatment.

### Culturing of the Parasites

Leishmania infantum (MHOM/MA/67/ITMA-P263) promastigotes are incubated at 27° C. in RPMI medium (with heat inactivated 10% fetal bovine serum, 2 mM L-glutamine, 20 mM HEPES, 100 U/ml penicillin, 100 μg/ml streptomycin). Parasites reaching the logarithmic phase (10⁶/ml) are made infective.

### Culturing of the Macrophages

Macrophage J774 cell line (ATCC) is grown as a single layer in heat-inactivated RPMI 1640 nutrient medium with 10% FBS (2 mM L-glutamine, 100 U/ml penicillin, 100 μg/ml streptomycin) in a humidified atmosphere of 5% CO₂ at 37° C.) and the cells are passaged at 72 hour intervals.

Infecting Macrophage Cells with Leishmania Infantum Parasites

The macrophages are infected with parasites at a ratio of 10:1 (parasite: macrophage) at 37° C. and after 5 hours, the infected macrophages are washed with the medium to remove any residual parasites. Then they are fixed and stained with Giemsa and the percentage of infection is determined according to the following criterion:

$$\frac{the number of macrophage cells infected with the parasite}{total number of macrophage cells} \times 100$$

Collecting the Medium of the Macrophage Cells Infected with Leishmania infantum Parasites

The extracellular vesicles obtained from the macrophage cells infected with parasites are isolated by an isolation method selected from the group comprising isolation by two-phase liquid system, graduated centrifuge, ultrafiltration, chromatographic methods, polymer based isolation and isolation by microbeads. Among them, the purest extracellular vesicle isolation is achieved by isolation with two-phase liquid system and therefore this isolation method is preferred within the scope of the present application.

### Isolation of the Extracellular Vesicles

collecting culture media of the parasite from which the extracellular vesicles will be isolated,
centrifuging at a rate of 2,000 g to 10,000 g for 5-20 minutes for removal of the undesirable substances such as cell residues and parasites from the culture media,
removing the particles sized 220 nm and above by filtration after centrifugation,
transferring the vesicle-protein mixture obtained by centrifugation into a two-phase liquid system containing PEG phase and DEX phase for separation thereof,
removing the nonvesicular proteins, cellular fat and other impurities from the vesicles by utilizing the chemical tendency of the PEG phase to the proteins and the DEX phase to the phospholipid structured membranes,
obtaining the isolated vesicles.
Particle concentration of the isolated exosomes is analyzed by NanoSight device.
   Loading Amphotericin B on Exosomes Obtained from Macrophages Infected with Leishmania infantum Parasites and Designating the Amount of the Encapsulated Substance

Amphotericin B is treated with exosomes at room temperature for half an hour and amphotericin B that is not loaded into the exosome is separated by centrifugation. Autofluorescent properties of Amphotericin B are utilized in designation of the amount. The amount of encapsulation of amphotericin B, whose absorbance value was read at 385 nm, was determined by UV spectrophotometer.

### Immunotherapeutic Use of Exosomes: Resazurin Assay

Leishmania parasites are incubated in culture medium and the effect of promastigotes of amphotericin B-loaded exosomes on proliferation is analyzed. In brief Leishmania infantum (MHOM/MA/67/ITMA-P263) promastigotes are incubated at 27° C. in RPMI medium (with heat inactivated 10% fetal bovine serum, 2 mM L-glutamine, 20 mM HEPES, 100 U/ml penicillin, 100 μg/ml streptomycin). Parasites (10⁶/ml) reaching the logarithmic phase—after 2 days of incubation—are incubated at 27° C. for 3 days with Amphotericin B and Amphotericin B-loaded exosome formulations at different concentration ranges. The viability of the parasites is determined by applying the Alamar Blue assay. Activities of the drug and drug-exosome formulations are found by reaching IC50 values from samples whose fluorescence intensities are read according to Alamar blue assay protocol.

Immunotherapeutic Use of Exosomes: Treating the Infected Macrophages with Amphotericin B-Loaded Infected Macrophage Exosomes

The effect of Amphotericin B-loaded exosomes on the proliferation of macrophage cells infected with parasites is analyzed. In brief, the macrophages are infected with parasites at a ratio of 10:1 (parasite:macrophage) at 37° C. After 5 hours, the infected macrophages are washed with the medium to remove any residual parasites and they are fixed and stained with Giemsa and the percentage of infection is determined according to the following criterion:

$$\frac{the number of macrophage cells infected with the parasite}{total number of macrophage cells} \times 100$$

The infected macrophages are incubated at 37° C. for 3 days with Amphotericin B and Amphotericin B-loaded exosome formulations at different concentration ranges. In order to determine the infection rate, they are fixed and stained with Giemsa and the percentage of infection is determined according to the following criterion:

$$\frac{the number of macrophage cells infected with the parasite}{total number of macrophage cells} \times 100$$

Prophylactic Use of Exosomes: Treating the Macrophages with the Macrophage Exosomes Infected with the Parasites

After the cells were seeded in 96-well culture plates (Corning Glasswork, Corning, N.Y.) at 20,000 cells/well in Dulbecco's modified Eagle's medium (DMEM) containing 10% fetal bovine serum (Invitrogen) and 1% PSA (Biological Industries, Beit Haemek, Israel) in the culture medium, they were treated with exosomes and the viability levels of the cells were measured on day 1, 2 and 3. Cell viability is measured by using 3-(4,5-di-methyl-thiazol-2-yl)-5-(3-carboxy-methoxy-phenyl)-2-(4-sulfo-phenyl)-2H-tetrazolium (MTS)-method (CellTiter96 AqueousOne Solution; Promega, Southampton, UK). 10 μl MTS solution is added onto the cells within a 100 μl growth medium and they are incubated in dark for 2 hours. After the incubation process, viability analysis is obtained by performing absorbance measurement via ELISA plate reader (Biotek, Winooski, Vt.) device at 490 nm wavelength.

Prophylactic Use of Exosomes: Treating the Macrophages with the Macrophage Exosomes Infected with the Parasites and then, after Infecting them with Parasites, Determining the Infection Rates

After being treated with the exosomes, the macrophages are infected with parasites at a ratio of 10:1 (parasite:macrophage) at 37° C. After 5 hours, the infected macrophages are washed with the medium to remove any residual parasites and they are fixed and stained with Giemsa and the percentage of infection is determined according to the following criterion:

$$\frac{the number of macrophage cells infected with the parasite}{total number of macrophage cells} \times 100.$$

What is claimed is:

1. Extracellular vesicles obtained from a culture medium of macrophages infected with parasites and used for Leishmaniasis as immunotherapeutic and prophylactic agents.

2. The extracellular vesicles according to claim 1, wherein the extracellular vesicles are obtained from the macrophages infected with at least one parasite selected from the group consisting of Leishmania spp. (L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, L. venezuelensis), Plasmadium spp. (P. falciparum, P. vivax, P. ovale), Schistosoma spp., Toxoplasma spp. (Toxoplasma gondii), and Trypanosoma brucei ssp.

3. The extracellular vesicles according to claim 2, wherein Leishmania infantum is selected as a parasite infecting the macrophages.

4. The extracellular vesicles according to claim 1, wherein the extracellular vesicles are isolated by at least one of the following methods: an isolation with aqueous two-phase systems (ATPS), a graduated centrifuge, an ultracentrifuge, a sucrose gradient ultracentrifuge, a polymeric precipitation, an ultrafiltration, an isolation with an antibody-affinity chromatography, an isolation with a peptide-affinity chromatography, a size separation chromatography, an isolation with microbeads, a precipitation according to ionic charges, and a salting.

5. The extracellular vesicles according to claim 4, wherein the extracellular vesicles are isolated by a two-phase liquid system comprising steps of
collecting culture media of a parasite, wherein the extracellular vesicles are isolated from the parasite,
centrifuging at a rate of 2,000 g to 10,000 g for 5 minutes-20 minutes for a removal of cell residues and the parasite from the culture media,
removing particles sized 220 nm and above by a filtration after the centrifuging to obtain a vesicle-protein mixture,
transferring the vesicle-protein mixture obtained by the centrifuging into the two-phase liquid system comprising a PEG phase and a DEX phase for a separation,
removing nonvesicular proteins, a cellular fat, and other impurities from the extracellular vesicles by utilizing a chemical tendency of the PEG phase to the nonvesicular proteins and the DEX phase to phospholipid structured membranes, and
obtaining isolated extracellular vesicles.

6. A pharmaceutical composition comprising the extracellular vesicles obtained from the macrophages infected with the parasites according to claim 1 and comprising at least one nano-carrier system selected from the group consisting of emulsion systems, biological and polymeric nanoparticles, biological and solid lipid nanoparticles, metallic nanoparticles, lipid vesicular systems including liposomes, niosomes, and ethosomes, dendrimers, polymer-drug conjugates, micelles, and carbon nanotubes.

7. The pharmaceutical composition according to claim 6, comprising at least one active compound selected from the group consisting of active compounds showing an antiparasitic activity and/or an antineoplastic activity, a binary combination of the antiparasitic activity and the antineoplastic activity, and a ternary combination of the antiparasitic activity and the antineoplastic activity as an active substance.

8. The pharmaceutical composition according to claim 7, comprising at least one agent selected from the group consisting of nitazoxanide, melarsoprol, eflornithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, ornidazole, paromomycin sulfate, pentamidine, pirimethamine, fenbendazole, triclabendazole, flubendazole, abamectin, suramin, levamisole, oxyclozanide, monepantel, derquantel, urea stibamine, sodium stibogluconate, meglumine antimoniate, paromomycin, fluconazole, a binary combination of the nitazoxanide, the melarsoprol, the eflornithine, the metronidazol, the tinidazole, the miltefosine, the mebendazole, the pyrantel pamoate, the thiabendazole, the diethylcarbamazine, the ivermectin, the niclosamide, the praziquantel, the albendazole, the rifampin, the amphotericin B, the fumagillin, the furazolidone, the nifursemizone, the ornidazole, the paromomycin sulfate, the pentamidine, the pirimethamine, the fenbendazole, the triclabendazole, the flubendazole, the abamectin, the suramin, the levamisole, the oxyclozanide, the monepantel, the derquantel, the urea stibamine, the sodium stibogluconate, the meglumine antimoniate, the paromomycin, and the fluconazole, a ternary combination of the nitazoxanide, the melarsoprol, the eflornithine, the metronidazol, the tinidazole, the miltefosine, the mebendazole, the pyrantel pamoate, the thiabendazole, the diethylcarbamazine, the ivermectin, the niclosamide, the praziquantel, the albendazole, the rifampin, the amphotericin B, the fumagillin, the furazolidone, the nifursemizone, the ornidazole, the paromomycin sulfate, the pentamidine, the pirimethamine, the fenbendazole, the triclabendazole, the flubendazole, the abamectin, the suramin, the levamisole, the oxyclozanide, the monepantel, the derquantel, the urea stibamine, the sodium stibogluconate, the meglumine antimoniate, the paromomycin, and the fluconazole, and encapsulations of the nitazoxanide, the melarsoprol, the eflornithine, the metronidazol, the tinidazole, the miltefosine, the mebendazole, the pyrantel pamoate, the thiabendazole, the diethylcarbamazine, the ivermectin, the niclosamide, the praziquantel, the albendazole, the rifampin, the amphotericin B, the fumagillin, the furazolidone, the nifursemizone, the ornidazole, the paromomycin sulfate, the pentamidine, the pirimethamine, the fenbendazole, the triclabendazole, the flubendazole, the abamectin, suramin, the levamisole, the oxyclozanide, the monepantel, derquantel, the urea stibamine, the sodium stibogluconate, the meglumine antimoniate, the paromomycin, and the fluconazole as an active compound showing the antiparasitic activity.

9. The pharmaceutical composition according to claim 7, comprising at least one agent selected from the group consisting of cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxiprogesterone, octreotide, tamoxifen, bendamustine, carmustine, chlorambucil, lomustine, melphalan, procarbazine, streptozocin, fludarabine, raltitrexed, actinomycin D, dactinomycin, doxorubicin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, vincristine, a binary combination of the cyclophosphamide, the ifosfamide, the temozolomide, the capecitabine, the 5-fluorouracil, the methotrexate, the gemcitabine, the pemetrexed, the mitomycin, the bleomycin, the epirubicin, the doxorubicin, the etoposide, the paclitaxel, the irinotecan, the docetaxel, the vincristine, the carboplatin, the cisplatin, the oxaliplatin, the bevacizumab, the cetuximab, the gefitinib, the imatinib, the trastuzumab, the denosumab, the rituximab, the sunitinib, the zoledronate, the abiraterone, the anastrozole, the bicalutamide, the exemestane, the goserelin, the medroxiprogesterone, the octreotide, the tamoxifen, the bendamustine, the carmustine, the chlorambucil, the lomustine, the melphalan, the procarbazine, the streptozocin, the fludarabine, the raltitrexed, the actinomycin D, the dactinomycin, the doxorubicin, the mitoxantrone, the eribulin, the topotecan, the vinblastine, the vinorelbine, the afatinib, the aflibercept, the crizotinib, the dabrafenib, the interferon, the ipilimumab, the lapatinib, the nivolumab, the panitumumab, the pembrolizumab, the pertuzumab, the sorafenib, the trastuzumab emtansine, the temsorilimus, the vemurafenib, the ibandronic acid, the pamidronate, the bexarotene, the buserelin, the cyproterone, the degarelix, the folinic acid, the fulvestrant, the lanreotide, the lenalidomide, the letrozole, the leuprorelin, the megestrol, the mesna, the thalidomide, and the vincristine, a ternary combination of the cyclophosphamide, the ifosfamide, the temozolomide, the capecitabine, the 5-fluorouracil, the methotrexate, the gemcitabine, the pemetrexed, the mitomycin, the bleomycin, the epirubicin, the doxorubicin, the etoposide, the paclitaxel, the irinotecan, the docetaxel, the vincristine, the carboplatin, the cisplatin, the oxaliplatin, the bevacizumab, the cetuximab, the gefitinib, the imatinib, the trastuzumab, the denosumab, the rituximab, the sunitinib, the zoledronate, the abiraterone, the anastrozole, the bicalutamide, the exemestane, the goserelin, the medroxiprogesterone, the octreotide, the tamoxifen, the bendamustine, the carmustine, the chlorambucil, the lomustine, the melphalan, the procarbazine, the streptozocin, the fludarabine, the raltitrexed, the actinomycin D, the dactinomycin, the doxorubicin, the mitoxantrone, the eribulin, the topotecan, the vinblastine, the vinorelbine, the afatinib, the aflibercept, the crizotinib, the dabrafenib, the interferon, the ipilimumab, the lapatinib, the nivolumab, the panitumumab, the pembrolizumab, the pertuzumab, the sorafenib, the trastuzumab emtansine, the temsorilimus, the vemurafenib, the ibandronic acid, the pamidronate, the bexarotene, the buserelin, the cyproterone, the degarelix, the folinic acid, the fulvestrant, the lanreotide, the lenalidomide, the letrozole, the leuprorelin, the megestrol, the mesna, the thalidomide, and the vincristine, and encapsulations thereof the cyclophosphamide, the ifosfamide, the temozolomide, the capecitabine, the 5-fluorouracil, the methotrexate, the gemcitabine, the pemetrexed, the mitomycin, the bleomycin, the epirubicin, the doxorubicin, the etoposide, the paclitaxel, the irinotecan, the docetaxel, the vincristine, the carboplatin, the cisplatin, the oxaliplatin, the bevacizumab, the cetuximab, the gefitinib, the imatinib, the trastuzumab, the denosumab, the rituximab, the sunitinib, the zoledronate, the abiraterone, the anastrozole, the bicalutamide, the exemestane, the goserelin, the medroxiprogesterone, the octreotide, the tamoxifen, the bendamustine, the carmustine, the chlorambucil, the lomustine, the melphalan, the procarbazine, the streptozocin, the fludarabine, the raltitrexed, the actinomycin D, the dactinomycin, the doxorubicin, the mitoxantrone, the eribulin, the topotecan, the vinblastine, the vinorelbine, the afatinib, the aflibercept, the crizotinib, the dabrafenib, the interferon, the ipilimumab, the lapatinib, the nivolumab, the panitumumab, the pembrolizumab, the pertuzumab, the sorafenib, the trastuzumab emtansine, the temsorilimus, the vemurafenib, the ibandronic acid, the pamidronate, the bexarotene, the buserelin, the cyproterone, the degarelix, the folinic acid, the fulvestrant, lanreotide, the lenalidomide, the letrozole, the leuprorelin, the megestrol, the mesna, the thalidomide, and the vincristine as an active compound showing the antineoplastic activity in a combination with the extracellular vesicles and/or the nano-carrier systems.

10. The pharmaceutical composition according to claim 6, comprising the extracellular vesicles obtained from the macrophages infected with the parasites in combination with at least one of aluminum hydroxide, aluminum phosphate, tocopherol emulsion systems containing 3D-MPL, cholesterol, and CG oligonucleotide.

11. The pharmaceutical composition according to claim 6, wherein a method of an administration for a treatment comprises at least one selected from the group consisting of parenteral, intravenous, intradermal, subcutaneous, intraperitoneal, topical, intrathecal, intranasal, intracerebroventricular, ocular, vaginal, urethral, transdermal, sublingual, subarachnoid, rectal, periodontal, perineural, peridural, periarticular, oral, intratympanic, intratumor, intrapulmonary, intrasynovial, intramuscular, intraovarian, intrameningeal, intracorporus cavernosum, intracoronary, intracerebral, epidural, cutaneous, buccal, and dental.

12. A pharmaceutical composition for a use in a treatment of Leishmaniasis as an adjuvant according to claim 6, wherein the pharmaceutical composition is formed by incorporating the extracellular vesicles obtained from the macrophages infected with the parasites into at least one of aluminum hydroxide, aluminum phosphate, tocopherol emulsion systems containing 3D-MPL, cholesterol, and CG oligonucleotide.

13. The extracellular vesicles according to claim 2, wherein the extracellular vesicles are isolated by at least one of the following methods: an isolation with aqueous two-phase systems (ATPS), a graduated centrifuge, an ultracentrifuge, a sucrose gradient ultracentrifuge, a polymeric precipitation, an ultrafiltration, an isolation with an antibody-affinity chromatography, an isolation with a peptide-affinity chromatography, a size separation, an isolation with microbeads, a precipitation according to ionic charges, and a salting.

14. The extracellular vesicles according to claim 3, wherein the extracellular vesicles are isolated by at least one of the following methods: an isolation with aqueous two-phase systems (ATPS), a graduated centrifuge, an ultracentrifuge, a sucrose gradient ultracentrifuge, a polymeric precipitation, an ultrafiltration, an isolation with an antibody-affinity chromatography, an isolation with a peptide-affinity chromatography, a size separation, an isolation with microbeads, a precipitation according to ionic charges, and a salting.

15. The extracellular vesicles according to claim 13, wherein the extracellular vesicles are isolated by a two-phase liquid system comprising steps of
collecting culture media of a parasite, wherein the extracellular vesicles are isolated from the parasite,
centrifuging at a rate of 2,000 g to 10,000 g for 5 minutes-20 minutes for a removal of cell residues and the parasite from the culture media,
removing particles sized 220 nm and above by a filtration after the centrifuging to obtain a vesicle-protein mixture,
transferring the vesicle-protein mixture obtained by the centrifuging into the two-phase liquid system comprising a PEG phase and a DEX phase for a separation,
removing nonvesicular proteins, a cellular fat, and other impurities from the extracellular vesicles by utilizing a chemical tendency of the PEG phase to nonvesicular proteins and the DEX phase to phospholipid structured membranes, and
obtaining isolated extracellular vesicles.

16. Extracellular vesicles according to claim 14, wherein the extracellular vesicles are isolated by a two-phase liquid system comprising steps of
collecting culture media of the parasite, wherein the extracellular vesicles are isolated from the parasite,
centrifuging at a rate of 2,000 g to 10,000 g for 5 minutes-20 minutes for a removal of cell residues and the parasite from the culture media,
removing particles sized 220 nm and above by a filtration after the centrifuging to obtain a vesicle-protein mixture,
transferring the vesicle-protein mixture obtained by the centrifuging into the two-phase liquid system comprising a PEG phase and a DEX phase for a separation,
removing nonvesicular proteins, a cellular fat, and other impurities from the extracellular vesicles by utilizing a chemical tendency of the PEG phase to nonvesicular proteins and the DEX phase to phospholipid structured membranes, and
obtaining isolated extracellular vesicles.

17. The pharmaceutical composition according to claim 6, wherein the extracellular vesicles are obtained from the macrophages infected with at least one parasite selected from the group consisting of Leishmania spp. (L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, L. venezuelensis), Plasmadium spp. (P. falciparum, P. vivax, P. ovale), Schistosoma spp., Toxoplasma spp. (Toxoplasma gondii), and Trypanosoma brucei ssp.

18. The pharmaceutical composition according to claim 17, wherein Leishmania infantum is selected as a parasite infecting the macrophages.

19. The pharmaceutical composition according to claim 6, wherein the extracellular vesicles are isolated by at least one of the following methods: an isolation with aqueous two-phase systems (ATPS), a graduated centrifuge, an ultracentrifuge, a sucrose gradient ultracentrifuge, a polymeric precipitation, an ultrafiltration, an isolation with an antibody-affinity chromatography, an isolation with a peptide-affinity chromatography, a size separation, an isolation with microbeads, a precipitation according to ionic charges, and a salting.

20. The pharmaceutical composition according to claim 19, wherein the extracellular vesicles are isolated by a two-phase liquid system comprising steps of
collecting culture media of a parasite, wherein the extracellular vesicles are isolated from the parasite,
centrifuging at a rate of 2,000 g to 10,000 g for 5 minutes-20 minutes for a removal of cell residues and the parasite from the culture media,
removing particles sized 220 nm and above by a filtration after the centrifuging to obtain a vesicle-protein mixture,
transferring the vesicle-protein mixture obtained by the centrifuging into the two-phase liquid system comprising a PEG phase and a DEX phase for a separation,
removing nonvesicular proteins, a cellular fat, and other impurities from the extracellular vesicles by utilizing a chemical tendency of the PEG phase to nonvesicular proteins and the DEX phase to phospholipid structured membranes, and
obtaining isolated extracellular vesicles.
